(19) United States
(12) Patent Application Publication
(10) Pub. No.: US 2016/0008233 A1
(43) Pub. Date: Jan. 14, 2016

(54) DENTAL ALLOY

(71) Applicant: DeguDent GmbH, Hanau (DE)

(72) Inventors: Joerg HACHENBERG, Aschaffenberg (DE); Rudi STEINKE, Hanau (DE); Angela KLAUS, Hanau (DE); Irmgard WISSEL, Freigericht (DE)

(21) Appl. No.: 14/865,340

(22) Filed: Sep. 25, 2015

Related U.S. Application Data
(63) Continuation of application No. 13/070,997, filed on Mar. 24, 2011, now abandoned.

(30) Foreign Application Priority Data
Mar. 26, 2010 (DE) 102010016171.3
Jul. 20, 2010 (EP) 10170137.3

(51) Int. Cl. A61K 6/04; C22C 30/04; C22C 30/06; C22C 30/02

(52) U.S. Cl. CPC A61K 6/046; C22C 30/02; C22C 30/04; C22C 30/06; A61K 6/04

(57) ABSTRACT

A palladium-dominated dental alloy, in particular a ceramic-bonding dental alloy for the manufacture of dental prostheses such as crowns, bridges, inlays, or onlays, containing at least gold, palladium, and silver, as well as a grain-growth inhibitor in the form of ruthenium. In order to achieve a fine-grained separation without the formation of agglomerates to obtain a dental alloy with high mechanical stability and excellent polishing characteristics, it is proposed that the dental alloy contain—in addition to ruthenium as grain-growth inhibitor—at least one element of the group tantalum, niobium, yttrium, zirconium, chromium, and molybdenum as grain-refinement control element.

### BACKGROUND OF THE INVENTION

The invention relates to a palladium-dominated dental alloy, in particular a ceramic-bonding dental alloy for the manufacture of dental prostheses such as dental crowns, bridges, inlays, or onlays, containing at least gold, palladium, and silver, as well as a grain-growth inhibitor in the form of ruthenium.

A dental alloy in accordance with DE-C-32 11 703 contains in % by weight: gold 10-60%, palladium 20-60%, and silver 0-15%. It further contains 0-10% indium, 0-10% tin, 0-5% zinc, 0-2% iridium, 0-2% copper, 0.1-5% platinum, and/or 0.05-2% of each of at least one of the transition elements of the 4^{th}, 5^{th}, and 6^{th} subgroup of the periodic table.

For the purpose of achieving high mechanical stability, in particular reproducible high-temperature stability in a palladium- and copper-free dental alloy with high gold content, EP-B-1 799 873 prescribes adding to the alloy a single grain-growth inhibitor and specific further elements that in combination result in a reduction of undesirable grain agglomerates. As grain-growth inhibitor one uses iridium or rhodium, whereby one uses iridium if niobium is present in the alloy, rhodium if tantalum is present, and iridium or rhodium if titanium or vanadium are present.

Also known are so-called reduced-gold-content palladium alloys, in which the palladium content in atomic percent is always greater than the gold content, so that these are referred to as palladium-dominated alloys, even though the gold content expressed in % by weight can be greater than the palladium content. Corresponding palladium-dominated alloys may contain ruthenium as grain-growth inhibitor. It has been discovered however, that ruthenium does not provide any controlled grain refinement.

A low-gold-content porcelain-bonding dental noble-metal alloy in accordance with DE-B-28 28 304 contains—in addition to gold, palladium, and silver—a titanium content of between 0.05 and 0.5%.

DE-C-29 44 755 discloses a porcelain-bonding dental alloy with a ruthenium content of between 0.175 and 0.25% by weight and a silver content of between 5.86 and 11% by weight.

A dental alloy according to EP-A-0 057 149 contains in % by weight up to 20% silver, up to 40% palladium, up to 20% platinum, and up to 30% gold. It may further contain up to 3% by weight ruthenium, iridium, and rhodium with a content of zinc of up to 5% by weight and copper of up to 8% by weight. The alloy itself contains neither iridium nor tin. Allergenic metals such as iron, cobalt, nickel, chromium, and manganese are present with a proportion of less than 0.05% by weight.

A noble-metal alloy according to DE-A-31 46 794 necessarily contains gallium.

In a gold-coloured palladium-indium alloy in accordance with WO-A-90/07018 the gold content is at most 30% by weight.

A dental alloy according to US-A-2002/0122741 contains 15-75% Ag, up to 55% Au, 10-50% Pd, 6-25% Pt, and may contain In, Ga, Sn, Ge, Zn, Mn, Ir, Ru, Rh, Re, whereby the content of Ir, Ru, Rh, Re is no more than 3% for each element and for the combined total.

Palladium master alloys according to DE-A-38 30 666 contain a gold content of up to 20% by weight as well as a gallium content of between 0.5 and 5% by weight.

### SUMMARY OF THE INVENTION

The objective of the present invention is to further develop a palladium-dominated alloy of the above-mentioned type in a way to be able to provide a fine-grained separation without the formation of agglomerates to obtain a dental alloy with high mechanical stability as well as excellent polishing characteristics.

To meet this objective we propose that the dental alloy contain—in addition to ruthenium as grain-growth inhibitor—at least one element of the group comprising tantalum, niobium, yttrium, zirconium, chromium, molybdenum, as grain-refinement control element, whereby the dental alloy consists essentially of (in % by weight):

| | |
|---|---|
| 0.03 to 1 | ruthenium |
| 0.03 to 2 | grain-refinement control element |
| 0 to 10 | tin |
| 0 to 10 | zinc |
| 0 to 10 | indium |
| 0 to 10 | copper |
| 0 to 1 | gallium |
| 0 to 10 | platinum |
| 0 to 2 | iridium |
| 0 to 2 | rhodium |
| 0 to 2 | germanium |
| 0 to 2 | aluminium |
| 0 to 2 | silicon |
| 0 to 2 | cerium |
| 0 to 2 | lanthanum |
| 0 to 2 | iron, |

with the remainder being gold, silver, palladium,
whereby the sum total of elements adds up to 100% by weight.

### DETAILED DESCRIPTION OF THE INVENTION

The invention's palladium-dominated dental alloy consequently contains at least gold, silver, palladium, ruthenium, as well as one grain-refinement control element and may consist exclusively of these elements.

The addition of at least one grain-refinement control element in accordance with the invention is beneficial since the grain-refinement control element together with the grain-growth inhibitor such as ruthenium has a phase diagram that possesses a eutectic point. This serves to stabilize the liquid phase that contains the grain-growth inhibitor such as ruthenium, the at least one grain-refinement control element, and the remaining constituents of the alloy. Compared to solidification in the absence of a grain-refinement control element, the grain-growth inhibitor such as ruthenium now separates at a lower temperature and in a finer mixture with the at least one grain-refinement control element. During the solidification of the other constituents, these separation products act as crystallization nuclei, facilitating an overall more fine-grained structure formation.

The preferred grain-refinement control element is tantalum. But niobium or a mixture of niobium and tantalum also deserve mention.

Irrespective thereof, it is intended preferentially that the dental alloy contain more than 30% by weight of gold, more than 35% by weight of palladium, more than 10% by weight of silver, and more than 5% by weight of tin. Tin is used to increase the mechanical strength of the alloy. As is well known, this is achieved by way of the addition of base components such as tin, zinc, indium, and copper, whereby the best results in the preferred composition range were obtained using tin. Platinum as well can result in increased mechanical strength to some degree, but it is very expensive.

Irrespective thereof, the addition of gallium also results in increased material strength, whereby gallium can have a negative effect on biocompatibility. Consequently, the alloy preferably has a low or zero gallium content.

In particular, the invention is characterized by a dental alloys that consists essentially of (in % by weight):

| | |
|---|---|
| 30-45 | gold |
| 35-50 | palladium |
| 10-25 | silver |
| 6-10 | tin |
| 0.03-1.0 | ruthenium |
| 0.03-2.0 | grain-refinement control element, |

whereby the tin portion can be substituted proportionally up to a limit of 5% by weight with at least one element of the group comprising zinc, indium, platinum, copper, or a combination thereof.

In this, the up to 5% by weight of at least one element of the group comprising zinc, indium, platinum, copper, or a combination thereof refer to the overall alloy. As an example: instead of a tin content of 6-10% by weight one can for example also use 5% by weight zinc, which reduces the tin content to 1-5% by weight.

The invention is further characterized by the dental alloy consisting essentially of (in % by weight) :

| | |
|---|---|
| 30-45 | gold |
| 35-50 | palladium |
| 10-25 | silver |
| 6-10 | tin |
| 0.03-1.0 | ruthenium |
| 0.03-2.0 | grain-refinement control element. |

A preferred palladium-dominated dental alloy consists essentially of (in % by weight)

| | |
|---|---|
| 35-40 | gold |
| 38-45 | palladium |
| 12-20 | silver |
| 6-10 | tin |
| 0.03-1.0 | ruthenium |
| 0.03-2.0 | grain-refinement control element. |

A preferred palladium-dominated dental alloy consists essentially of (in % by weight)

| | |
|---|---|
| 38-42 | gold |
| 38-42 | palladium |
| 12-14 | silver |
| 6-9 | tin |
| 0.05-1.0 | ruthenium |
| 0.05-2.0 | grain-refinement control element. |

It is particularly preferred that the dental alloy consist essentially of (in % by weight)

| | |
|---|---|
| 39.0-41.0 | gold |
| 39.0-41.0 | palladium |
| 12.0-13.0 | silver |
| 6.5-8.0 | tin |
| 0.05-0.5 | ruthenium and |
| 0.1-1.0 | tantalum or |
| 0.1-1.0 | niobium. |

Again, the sum total of elements adds up to 100% by weight.

In particular, the dental alloy is not to contain any gallium. The preferred platinum content is less than 6% by weight, particularly preferred less than 5% by weight.

A preferred composition of the dental alloy is characterized by (in % by weight) 40.0 Au, 39.8 Pd, 12.4 Ag, 7.5 Sn, 0.2 Ta, 0.1 Ru.

The invention proposes a palladium-dominated alloy, in which the chemical and metal-physical properties are determined by palladium, which in comparison to the state of technology produces finer grain structure and is free of agglomerates, which otherwise would have a negative effect on mechanical stability and polishing characteristics.

Added to this as constituents of the dental alloy are a grain-growth inhibitor and at least one grain-refinement control element, whereby the grain-growth inhibitor and the at least one grain-refinement control element have a phase diagram that possesses a eutectic point.

The temperature of the eutectic point in the binary phase diagram of the grain-growth inhibitor ruthenium and the grain-refinement control element preferably is more than 250° K below the lower solidification point of the pure elements, since a low eutectic point is desirable in this case. Naturally, it must be located above the melting temperature of the dental alloy. Thus, first to precipitate are the grain-growth inhibitor and the at least one grain-refinement control element, which form crystallization nuclei without having sufficient time for agglomeration to occur, which consequently allows formation of the desired fine grain structure.

The following table lists known alloys, alloys with a composition different from the one according to the invention, and alloys in accordance with the teaching of the invention. Inspection of micrographs revealed that dental alloys containing ruthenium as grain-growth inhibitor and tantalum or niobium as grain-refinement control element are fine-grained, whereby agglomerates having a negative effect on mechanical stability and polishing characteristics do not manifest at all or only to a negligible extent.

| | Composition in % by weight | | | | | | | | | | | | |
|---|---|---|---|---|---|---|---|---|---|---|---|---|---|
| Alloy | Pd | Au | Ag | Sn | In | Zn | Ga | Pt | Ru | Ta | Nb | Ir | Result |
| Known alloy I | 35.00 | 39.00 | 19.40 | 5.00 | 0.50 | — | — | 1.00 | 0.05 | — | — | 0.05 | Coarse-grained, highly |
| | | | | | | | | | | | | | segregated, pronounced |
| | | | | | | | | | | | | | agglomerates |
| Known alloy II | 39.40 | 40.00 | 10.00 | 0.20 | 8.80 | — | 1.40 | — | 0.20 | — | — | — | Coarse-grained, 2. |
| | | | | | | | | | | | | | Phase (high Ga content), |
| | | | | | | | | | | | | | pronounced |
| | | | | | | | | | | | | | agglomerates |
| Known alloy III | 35.60 | 40.00 | 17.60 | 5.00 | — | — | 0.50 | 1.00 | — | 0.10 | — | 0.20 | Coarse-grained, highly |
| | | | | | | | | | | | | | segregated, 2. |
| | | | | | | | | | | | | | Phase (high Ga content), |
| | | | | | | | | | | | | | some agglomerates |
| EHF10* | 39.80 | 40.00 | 12.02 | 8.00 | — | — | — | — | 0.09 | — | — | 0.09 | Relatively fine- |
| | | | | | | | | | | | | | grained, but highly |
| | | | | | | | | | | | | | pronounced agglomerates |
| EHF12* | 39.80 | 40.00 | 13.10 | 7.00 | — | — | — | — | 0.10 | — | — | — | Dentritic/coarse- |
| | | | | | | | | | | | | | grained, some agglomerates |
| EHF13 | 39.80 | 40.00 | 12.80 | 7.00 | — | — | — | — | 0.10 | — | 0.30 | — | Fine-grained, almost |
| | | | | | | | | | | | | | no agglomerates |
| EHF14 | 39.80 | 40.00 | 12.90 | 7.00 | — | — | — | — | 0.10 | 0.20 | — | — | Fine-grained, almost |
| | | | | | | | | | | | | | no agglomerates |
| EHF16 | 39.80 | 40.00 | 12.40 | 7.50 | — | — | — | — | 0.10 | 0.20 | — | — | Fine-grained, almost |
| | | | | | | | | | | | | | no agglomerates |
| EHF19 | 50.00 | 20.00 | 25.00 | — | 4.70 | — | — | — | 0.10 | 0.20 | — | — | Fine-grained, no |
| | | | | | | | | | | | | | agglomerates |

*experimental examples not in accordance with this invention

What is claimed is:

1. A palladium-dominated dental alloy, in particular a ceramic-bonding dental alloy for the manufacture of dental prostheses such as crowns, bridges, inlays, or onlays, consisting essentially of at least gold, palladium, and silver, as well as one grain-growth inhibitor in the form of ruthenium, wherein in addition to ruthenium as grain-growth inhibitor, the dental alloy contains at least one element selected from the group consisting of tantalum, niobium, yttrium, zirconium, chromium, and molybdenum as grain-refinement control element, whereby the dental alloy consists essentially of (in % by weight):

| | |
|---|---|
| 0.03 to 1 | ruthenium |
| 0.03 to 2 | grain-refinement control element |
| 0 to 10 | tin |
| 0 to 10 | zinc |
| 0 to 10 | indium |
| 0 to 10 | copper |
| 0 to 1 | gallium |
| 0 to 10 | platinum |
| 0 to 2 | iridium |
| 0 to 2 | rhodium |
| 0 to 2 | germanium |
| 0 to 2 | aluminium |
| 0 to 2 | silicon |
| 0 to 2 | cerium |
| 0 to 2 | lanthanum |
| 0 to 2 | iron, |

with the remainder being gold, palladium, and silver,
whereby the total sum of elements adds up to 100% by weight.

2. The palladium-dominated dental alloy of claim 1, wherein the dental alloy contains as grain-refinement control element tantalum or niobium, or tantalum and niobium.

3. The palladium-dominated dental alloy of claim 1, wherein the dental alloy contains more than 30% by weight of gold, more than 35% by weight of palladium, more than 10% by weight of silver, and more than 5% by weight of tin.

4. The palladium-dominated dental alloy of claim 1, wherein the dental alloy consists essentially of (in % by weight)

| | |
|---|---|
| 30-45 | gold |
| 35-50 | palladium |
| 10-25 | silver |
| 6-10 | tin |
| 0.03-1.0 | ruthenium |
| 0.03-2.0 | grain-refinement control element, |

whereby the tin content may be replaced proportionally up to 5% by weight with at least one element of the group comprising zinc, indium, platinum, copper or a combination thereof.

5. The palladium-dominated dental alloy of claim 1, wherein the dental alloy consists essentially of (in % by weight):

| | |
|---|---|
| 30-45 | gold |
| 35-50 | palladium |
| 10-25 | silver |
| 6-10 | tin |
| 0.03-1.0 | ruthenium |
| 0.03-2.0 | grain-refinement control element. |

6. The palladium-dominated dental alloy of claim 1, wherein the dental alloy consists essentially of (in % by weight):

| | |
|---|---|
| 35-40 | gold |
| 38-45 | palladium |
| 12-20 | silver |
| 6-10 | tin |
| 0.03-1.0 | ruthenium and |
| 0.03-2.0 | grain-refinement control element. |

7. The palladium-dominated dental alloy of claim 1, wherein the dental alloy consists essentially of (in % by weight):

| | |
|---|---|
| 38-42 | gold |
| 38-42 | palladium |
| 12-14 | silver |
| 6-9 | tin |
| 0.05-1.0 | ruthenium |
| 0.05-2.0 | grain-refinement control element, |

8. The palladium-dominated dental alloy of claim 1, wherein the dental alloy consists essentially of (in % by weight):

| | |
|---|---|
| 39.0-41.0 | gold |
| 39.0-41.0 | palladium |
| 12.0-13.0 | silver |
| 6.5-8.0 | tin |
| 0.05-0.5 | ruthenium and |
| 0.1-1.0 | tantalum or |
| 0.1-1.0 | niobium. |

9. The palladium-dominated dental alloy of claim 1, wherein the dental alloy consists essentially of (in % by weight):

| | |
|---|---|
| 40.0 | gold |
| 39.8 | palladium |
| 12.4 | silver |
| 7.5 | tin |
| 0.2 | tantalum |
| 0.1 | ruthenium. |

10. The palladium-dominated dental alloy of claim 1, wherein the gallium content is 0% by weight.

11. The palladium-dominated dental alloy of claim 1, wherein the platinum content is less than 6% by weight.

12. The palladium-dominated alloy of claim 11, wherein the platinum content is less than 5% by weight.
